(19) United States
(12) Patent Application Publication
(10) Pub. No.: US 2005/0287227 A1
(43) Pub. Date: Dec. 29, 2005

(54) Supplement containing carotenoid, nicotinamide, zinc, water soluble extract of uncaria species and method of the same

(76) Inventors: Ronald Pero, Lund (SE); Vincent C. Giampapa, Little Falls, NJ (US)

Correspondence Address: MELVIN K. SILVERMAN, 500 WEST CYPRESS CREEK ROAD, SUITE 500, FT. LAUDERDALE, FL, 33309, US

(21) Appl. No.: 11/106,200

(22) Filed: Apr. 14, 2005

Related U.S. Application Data
(60) Provisional application No. 60/562,967, filed on Apr. 16, 2004.

(51) Int. Cl. A61K 35/78; A61K 33/32

(52) U.S. Cl. 424/641; 424/769; 514/763; 514/356; 424/439

(57) ABSTRACT

A supplement for administering to a human, or other mammals, includes a carotenoid material, a nicotinamide material, a zinc source material, and a water soluble extract material of an Uncaria species. The supplement can be in a form for oral administration, particularly in a form of nutritional drink, or in a formulation for parenteral administration. Also disclosed is a method of treating a human including administering to an individual the supplement daily in amounts effective, in combination, to improve the individual's resistance to DNA damage, enhance DNA repair capacity, stimulate immune cell function, and inhibit tumor cell growth.

### CROSS REFERENCE TO RELATED APPLICATION

This application claims the benefit under 35 USC 119 (e) of the provisional patent application Ser. No. 60/562,967, filed on Apr. 16, 2004, which is hereby incorporated by reference in its entirety.

### FIELD OF THE INVENTION

This invention relates to a supplement for treating humans and other animals to reduce DNA damage, enhance DNA repair capacity, and enhance immune function, and the method of use. More particularly, it relates to liquid supplement containing a carotenoid material, a nicotinamide material, a zinc source material, a water soluble extract material of an Uncaria species and an aqueous medium.

### BACKGROUND OF THE INVENTION

The exact mechanism of action of carotenoids such as beta carotene is not fully understood but it is commonly accepted scientifically that one primary mechanism is to scavenge oxygen derived free radicals produced either as by-products of metabolism or from exogenous environmental exposures. As a free radical scavenger, carotenoids can be expected to reduce or protect against the chemical damage induced in DNA, RNA and protein of cells by toxic environmental exposures or endogenous cellular metabolic errors that ultimately can result in a disease state.

Nicotinamide and its metabolic equivalent nicotinic acid (niacin, vitamin B₃) or even tryptophane which is the synthetic precursor to niacin is the main precursor for the formation and maintenance of the cellular pool of nicotinamide adenine dinucleotide (NAD). NAD is essential for cellular ATP production and maintenance of the cell's redox potential, and it is also the substrate for the DNA repair enzyme, poly ADP-ribosyl transferase (ADPRT). Niacin deprivation decreases the NAD pool significantly both in tissue culture cells, animal systems and humans. The NAD depleted cells have an increased sensitivity to DNA damage, and the levels of poly(ADP-ribose) production in cultured cells or in rat liver were significantly lower after mild nicotinamide deficiency. On the other hand, when niacin was given as a supplement to ordinary nutrition (i.e. above known dietary levels) the NAD pool increased and the cells were less sensitive to oxygen radicals. Therefore, the primary mechanism of action of nicotinamide/niacin differs from carotenoids in that the cell's potential for energy metabolism is increased by amplifying NAD and ATP pool supplies (i.e. these biochemicals are the energy sources of living organisms) which in turn is useful to cells, tissues and organs to reduce DNA damage, enhance DNA repair (i.e. poly ADP-ribosylation) and stimulate immune function where the relevance to the disease state is apparent.

Zinc differs from the carotenoids and nicotinamide with regard to its mechanism of action in that it influences disease development and immune function by being an essential co-factor in several enzyme functions involving replication, DNA repair and antioxidant defense of cells. Zinc is required for cell replication and DNA polymerase activity. There are two zinc fingers in the DNA binding domain of the poly adenosine diphosphate ribosyl transferase (ADPRT) gene and other DNA repair proteins which contain cysteine residues, and if these cysteine residues are oxidized at their thiol constituents, they would prevent DNA binding and participation in DNA repair. Moreover, superoxide dismutase is an antioxidant enzyme protecting cells from the harmful superoxide anion because this radical is a substrate for the enzymatic reaction that also requires zinc as a cofactor.

Furthermore, U.S. Pat. No. 6,020,351 (to Pero) teaches the use of a combination of carotenoids, nicotinamide, and zinc, in the absence of other active components, to reduce DNA damage, enhance DNA repair capacity, and enhance immune function. Commercially, a combination of these three materials is available under the tradename of Nicoplex®.

As taught in U.S. Pat. Nos. 6,039,949, 6,238,675, 6,361,805 and copending patent application Ser. No. 10/093,794, the water soluble extract of an Uncaria species known as C-MED-100® or Activar AC-11™, hence its bioactive components, carboxy alkyl esters, is known to give profound nutritional support as a dietary supplement because the water soluble extract of an Uncaria species enhances DNA repair process and immune functions, which, in turn, are the critical physiological processes that regulate aging. Both of these processes involve regulating the nuclear transcription kappa beta (NF-kB). NF-kB is well known to control (i) the nuclear events that salvage cells from apoptotic cell death and (ii) pro-inflammatory cytokine production. (Beg, A A and Baltimore, D., An essential role for NF-kB in preventing TNF-α induced cell death. Science 274: 782-784, 1996; Wang, C-Y, Mayo, M. W., Baldwin, A. S., TNF-α and cancer therapy-induced apoptosis: Potentiation by inhibition of NF-KB. Science 274: 784-787, 1996). Hence, this mechanism directly connects induction of apoptosis to programmed cell toxicity with inhibition of pro-inflammatory cytokine production and inflammation. This is different from the mechanisms of the above-described three chemicals.

As shown above, each individual component has a different mechanism of action at cellular or molecular level for enhancing cell normal functions. However, in the prior art the above-described four materials have not been used, nor recognized, in a composition or as a system, to obtain further enhanced functions in improving an individual's resistance to DNA damage, enhancing DNA repairing capacity and immune function, and preventing aging related disorders.

On the other hand, it is known that acidic pH plays an important role in various pathophysiological states and has been demonstrated to be carcinogenic in animal models. Recent studies have also implicated acidic pH in the development of preneoplastic Barrett's esophagus in human. Recently, Xiao et al. have shown in the mouse skin carcinogenesis model study that application of acidic pH buffer (100 μl of 250 mM citrate phosphate, pH 2.5) to the mouse skin induces tumors in 9,10-dimethyl-1,2-benzanthracene(DMBA)-initiated mice (PNAS, April 2003, Vol. 100 No. 9, 5205). Furthermore, Xiao et al's studies in tissue culture models have suggested that acidic pH acts like a TOP2 poison VP-16 (demethylepipodophyllotoxin ethylidene-D-glucoside) to induce TOP2-mediated DNA damage: (i) acidic pH induces TOP2-dependent DNA damage signals as evidenced by up-regulation of p53 and Ser-139 phosphorylation of H2AX (a substrate for ataxia telangiectasia mutated (ATM)/ATM and Rad3-related (ATR) kinases); (ii) acidic pH-induced cytotoxicity in tumor cells is reduced in TOP2-deficient cells; (iii) acidic pH increases the mutation frequency of the hypoxanthine phosphoribosyl transferase (HPRT) gene in a TOP2-dependent manner; and (iv) acidic pH induces reversible TOP2-mediated DNA strand breaks in vitro. These scientific research results have strongly suggested the disadvantages of drinking acidic beverage, such as those popular carbonated soft drinks which typically has a pH below 4.

In the early 1950's, the Japanese developed water ionizers that split tap water into acidic and alkaline water. They discovered that alkaline water was beneficial for mammals, including humans. In 1966, the Health and Rehabilitation Ministry of the Japanese Government approved these types of water ionizers as health improvement medical devices. In another approach of providing alkaline water, U.S. Pat. No. 5,306,511 (to Whang) teaches alkaline additive for drinking water, which is a concentrate alkaline solution of a mixture of potassium hydroxide and sodium hydroxide. Whang further teaches an alkaline drinking water having a pH in a range from about 9 to 12, and a method of producing the alkaline drinking water by adding the additive into tap water.

Based on the above described, it is desirable to have a nutritional supplement containing a carotenoid material; a nicotinamide material; a zinc source material and the water soluble extract material of the Uncaria species, which has further enhanced functions in improving mammal's, particularly human's resistance to DNA damage, enhancing DNA repairing capacity and immune function, and preventing aging related disorders. It is desirable to provide such a supplement in a convenient form of nutritional drink which preferably has an alkaline pH.

### SUMMARY OF THE INVENTION

In one embodiment, the present invention is directed to a supplement for administering to a human or other mammals. The supplement consists essentially of a carotenoid material, a nicotinamide material, a zinc source material, and a water soluble extract material of an Uncaria species. The supplement can be in a form for oral administration, particularly in the form of a liquid supplement as a nutritional drink, or in a form for parenteral administration. Preferably, the liquid supplement has a pH in a range from about 7.5 to about 9.0.

In another embodiment, the present invention is directed to a method of treating a mammal, particularly a human, using the supplement of the present invention. The method comprises administering to the mammal a supplement consisting essentially of a carotenoid material, a nicotinamide material, a zinc source material, and a water soluble extract material of an Uncaria species, wherein the materials being administered to the mammal in daily dosage of amounts effective, in combination, to improve resistance to DNA damage, enhance DNA repair capacity, stimulate immune cell function, and inhibit tumor cell growth. Preferably, the daily dosage for treating a human includes from about 50 to 150 mg of carotenoid, from about 50 to about 150 mg of nicotinamide, from about 5 to about 50 mg of a zinc salt, from about 100 to about 1000 mg of the water soluble extract of the Uncaria species.

### BRIEF DESCRIPTION OF DRAWINGS

FIG. 1 shows the DNA repair expressed by percent single strand breaks (SSB %) upon irradiation of rats which have been treated with different supplements, as described in Example 1.

FIG. 2 shows NF-kB inhibition in 70Z/3 cells treated with different supplements, as described in Example 2.

FIG. 3 shows HL-60 growth inhibition of human leukemic HL-60 cells treated with different supplements, as described in Example 3.

### DETAILED DESCRIPTION OF THE INVENTION

In one embodiment, the present invention provides a supplement which consists essentially of a carotenoid material, a nicotinamide material, a zinc source material, and a water soluble extract material of an Uncaria species, administered orally to increase an individual's resistance to cellular DNA damage, enhance cellular DNA repair and stimulate immune cell functions in vivo. The basic principle of this invention is to combine substances with known properties to enhance DNA repair capacity, stimulate immune cell responsiveness, and inhibit tumor cell growth, but with differing mechanisms of action. More specifically, carotenoids are electrophilic scavenger of radicals produced endogenously by cells or exogenously by the environment. Nicotinamide is amplified source of energy via increased production of NAD or ATP. Zinc is an essential cofactor to antioxidant, replicative and DNA repair enzymes in cells. The water soluble extract material of an Uncaria species prevents free radical damage by NF-kB inhibition, induces differentiation and immune cell responsiveness by apoptosis, enhances DNA repair, and inhibit tumor cell growth, which in turn are the major factors related to aging. In combination, these four different mechanisms can synergistically achieve improvement of resistance to DNA damage, enhancement of DNA repair capacity and immune cell responsiveness, and prevention of aging related disorders.

The term “carotenoid material” as used herein means carotenoids, such as alpha carotene, beta carotene, gamma carotene, lycopene or combination thereof. The term “nicotinamide material” as used herein means including nicotinamide, niacin, tryptophane (an amino acid precursor to niacin synthesis), NAD (nicotinamide-adenine dinucleotide), NADH (reduced form of NAD), NADP (NAD phosphate), NADPH (reduced form of NADP) or combination thereof. The term “zinc source material” as used herein means an appropriate source of zinc for administration to humans and/or other animals, e.g. one or more zinc salts, such as zinc sulfate or other zinc salts like amino acids such as methionine or aspartate, dipeptides, gluconates, halides, nitrates, oxides or acetates.

The Uncaria species includes tomentosa, guianensis, pteropoda, homomalla, perrottetii, or rhynchopylla. The term “water soluble extract of an Uncaria species” used herein refers to the water soluble extract of an Uncaria species obtained using the method described in U.S. Pat. Nos. 6,361,805, 6,238,675 and 6,039,949, which are hereby incorporated by reference in their entirety. Furthermore, the bioactive active component of the water soluble extract material of an Uncaria species has been identified as carboxy alkyl esters, as described in co-pending patent application Ser. No. 10/093,794, which is hereby incorporated by reference in its entirety.

The water soluble extract of an Uncaria species is commercially available under the product name Activar AC-11™, or C-Med-100®, from Optigenex, Inc, New York, N.Y. More specifically, Activar AC-11™ is a hot water extract from the bark of Uncaria tomentosa, produced according to the process described in U.S. Pat. No. 6,039,949. Briefly, the extract is produced from heating 150 gm of bark in 5 liters of tap water for 12 hours at 95° C., decanting the soluble fraction, ultra-filtrating the resulting water extract to remove all components having molecular weight larger than 10,000. The fraction having molecular weight less than 10,000 is spray dried. The product is in a form of beige to brown-orange hygroscopic fine powder, and it contains no less than 16% of carboxy alkyl esters, less than 0.05% of indole alkaloids (<10,000 Daltons) and 0% of indole alkaloids (>10,000 Daltons), and it is readily soluble in water (solubility in water >400 mg/ml).

In illustrative or preferred embodiment of the present invention, the carotenoid material can be alpha carotene, beta carotene, gamma carotene, Iycopene and mixtures thereof; the nicotinamide material can be nicotinamide, niacin, tryptophane or mixtures thereof; and the zinc source material may be one or more zinc salts. These materials can be obtained commercially. Two available suppliers are C. E. Jamieson, Ltd. (Ontario, Canada) and Integrated Biopharma, Inc. (Hillside, N.J.). The carotenoids can be supplied as Caroplex in 100 mg soft gel capsules or as water solubilized forms of lycopene or beta carotene. Nicotinamide is in 100 mg tablets and zinc gluconate is in 10 mg tablets, or Phytozinc®, a product of Nucycle Therapy, Inc. (Hillside, N.J.). Caroplex is a manufactured natural source of carotenoids from palm oil containing beta carotene=60%, alpha carotene=34%, gamma carotene=3% and Iycopene=3%. Other forms and dosages of these three materials are also available.

The concentrations of the active components in the supplement can be in broad ranges depending on the forms of the product. In one embodiment, the supplement can have from about 5 to about 100 mg of carotenoid, from about 5 to about 100 mg of nicotinamide, from about 1 to about 30 mg of a zinc salt, from about 10 to about 600 mg of the water soluble extract of an Uncaria species in the form of Activar AC-11™ in 100 ml of a pharmaceutically acceptable liquid carrier.

The supplement can be in various dosage forms, such as for example hard or soft-gelatin capsules, tablets, or powders, or in liquid dosage forms, such as elixirs, syrups, dispersed powders or granules, emulsions, or aqueous or oily suspensions. It can also be administered parenterally, in sterile liquid dosage forms.

Compositions intended for oral use can be prepared according to the methods known in the art for the manufacture of pharmaceutical compositions and such compositions may contain one or more agents including sweetening agents, flavoring agents, coloring agents, and preserving agents in order to provide a pharmaceutically elegant and palatable preparation.

Tablets contain the active components in admixture with non-toxic pharmaceutically acceptable excipients which are suitable for the manufacture of tablets. Such excipients include, for example, inert diluents, such as calcium phosphate, calcium carbonate, sodium carbonate, sodium phosphate, or lactose; granulating disintegrating agents, for example, maize starch or alginic acid; binding agents, such as starch, gelatin, or acacia; and lubricating agents, for example, magnesium stearate, stearic acids or talc. Compressed tablets may be uncoated or may be sugar coated or film coated by known techniques to mask any unpleasant taste and protect the tablet from the atmosphere, or enteric coated for selective disintegration and adsorption in the gastrointestinal tract.

Hard gelatin capsules or liquid filled soft gelatin capsules contain the active components and inert powdered or liquid carriers, such as, but not limited to calcium carbonate, calcium phosphate, kaolin, lactose, lecithin starch, cellulose derivatives, magnesium stearate, stearic acid, arachis oil, liquid paraffin, olive oil, pharmaceutically-accepted synthetic oils and other diluents suitable for the manufacture of capsules. Both tablets and capsules can be manufactured as sustained release-products to provide for continuous release of medication over a period of hours.

In a further embodiment, the present invention provides a liquid supplement which consists essentially of a carotenoid material, a nicotinamide material, a zinc source material, a water soluble extract material of an Uncaria species and an aqueous medium, as a nutritional drink, administered orally to increase an individual's resistance to cellular DNA damage, enhance cellular DNA repair and stimulate immune cell responsiveness in vivo.

The liquid supplement can be prepared according to those methods known in the art for the manufacture of beverage. The carotenoid material, nicotinamide material, zinc source material, and the water soluble extract material of an Uncaria species can be dissolved or suspended in an aqueous medium to produce a concentrate liquid supplement or a nutritional drink. As a nutritional drink, the concentrations of the active components in the liquid supplement can be at the lower portion of the concentration ranges described above, since a consumer can have multiple drinks a day.

Preferably, in the form of nutritional drink, the liquid supplement has an alkaline pH in a range from about 7.5 to about 9. The pH can be obtained by adjusting pH of the liquid supplement using a pharmaceutically acceptable pH adjusting agent, or a buffer. Suitable examples of pH adjusting agents and buffer include, but not limited to, sodium or potassium hydroxide, sodium or potassium carbonate, and phosphate buffer.

Moreover, the liquid supplement can also be in the form of aqueous suspensions in admixture with excipients suitable for the manufacture of aqueous suspensions. Such excipients are suspending agents, e.g., maltodextrin, sodium carboxymethylcellulose, methylcellulose, hydroxypropylmethylcellulose, sodium alginate, polyvinylpyrrolidone, gum tragacanth, and gum acacia; dispersing or wetting agents, such as a naturally occurring phosphatide, e.g., lecithin, or condensation products of an alkylene oxide with fatty acids, for example of polyoxyethylene stearate, or a condensation products of ethylene oxide with long chain aliphatic alcohols, e.g., heptadecaethyleneoxycetanol, or condensation products of ethylene oxide with partial esters derived from fatty acids and a hexitol, e.g., polyoxyethylene sorbitol monooleate, or a condensation product of ethylene oxide with partial esters derived from fatty acids and hexitol anhydrides, e.g., polyoxyethylene sorbitan monooleate. The aqueous suspensions can also contain one or more preservatives, for example ethyl, n-propyl, or p-hydroxy benzoate, one or more coloring agents, one or more flavoring agents, and one or more sweetening agents, such as sucrose, saccharin, or sodium or calcium cyclamate.

In a further embodiment, the present invention provides method of using the supplement to reduce DNA damage, improve DNA repairing processes, enhance immune function, and prevent aging related disorders.

The preferred daily dosage of the supplement is from about 50 to 150 mg of carotenoid, from about 50 to about 150 mg of nicotinamide, from about 5 to about 50 mg of a zinc salt, from about 100 to about 1000 mg of the water soluble extract of an Uncaria species in the form of Adtivar AC-11™ Examples 1 to 3 illustrated the effects of using the supplement of the present invention to improve resistance to DNA damage via enhanced DNA repair capacity thereby stimulating immune function. These, in turn, can lead to a reduction of endogenous oxidative which is generally believed to be the primary cause of aging, and can result in the reduction of aging related diseases.

The following examples are illustrative of the invention and are in no way to be interpreted as limiting the scope of the invention, as defined in the claims. It will be understood that various other ingredients and proportions may be employed, in accordance with the proceeding disclosure.

### EXAMPLE 1

### In Vivo Assessment of DNA Repair Estimated by Single Strand DNA Breaks (SSB) After 12 GY Gamma Irradiation Exposure

Four supplements were used in the test: (1) nicotinamide (abbreviated as NAM); (2) zinc (zinc gluconate); (3) carotenoids (lycopene and beta carotene); and (4) the commercial product Nicoplex® which contained 5% lycopene, 20% beta carotene, 13.3% zinc gluconate and 61.7% nicotinamide. The supplements were individually dissolved in either corn oil or water.

Five groups of Female W/Fu rats weighing 175-200 gm were used in the test. Four testing groups of rats were each administered orally by gavage one of the supplements, respectively, for 8 weeks on a daily basis. The control group was administered orally the liquid medium, corn oil or water. After 8 weeks on supplement, the rats were treated with 12 Gy of whole body irradiation in a 137 Cs source (Scanitronics, 1.56 Gy/min) and allowed to repair for 3 hours. The animals were then sacrificed. The spleen single-cell suspensions were prepared and then were frozen at −80° C. after addition of 10% dimethyl sulfoxide (DMSO). The frozen spleen cells were rapidly thawed before analysis at 37° C. by layering directly onto polycarbonate filters for evaluation of DNA single stranded breaks (SSB) using alkaline elution. The process has been described in detail by Olsson et al., Br J Can 74: 368-373, 1996, which is herein incorporated by reference in its entirety.

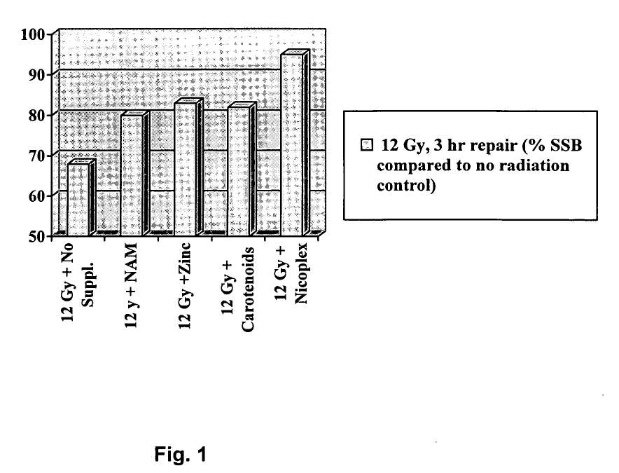

FIG. 1 shows the test results. As shown, in comparison to the control group, the rats treated by each of the four supplements described above illustrated a substantial improvement on the percent of DNA repair after being exposed to radiation. Particularly, the combination of three individual supplements in the form of Nicoplex® substantially increased DNA repair of the rats than the individual supplement functioning by itself.

These data indicated that when combining nicotinamide, zinc and carotenoid materials they did not metabolically compete with each other. Therefore, a combination of any two of these three materials, such as a combination of nicotinamide and zinc materials, a combination of nicotinamide and carotenoid materials, or a combination of zinc and carotenoid materials, can be utilized to enhance DNA repair.

### EXAMPLE 2

### In Vitro Analysis of NF-kB Activity

It is known that the mouse lymphoma 70Z/3 cell line has a recombined but transcriptionally silent immunoglobulin (Ig) k locus, the expression of which can be induced by activators such as lipopolysaccharide (LPS) (Sen and Baltimore Cell 47: 921-928, 1986). Ig light-chain expression leads to assembly of an Ig molecule that is expressed on the cell surface, and thus, surface staining of 70Z/3 cells for Ig expression presents a convenient measurement for NF-kB.

The commercial products Activar AC-11™, Nicoplex®, and their combination were used in the test. These products were dissolved in aqueous medium to produce three test supplement media: (1) 12 μg/ml of Activar AC-11™; (2) 136 μg/ml of Nicoplex®; and (3) 12 μg/ml of Activar AC-11™ plus 136 μg/ml of Nicoplex®.

The 70Z/3 cells were preincubated with each supplement medium, respectively, for 5 hours, and then they were subsequently treated with lipopoluysaccharride (Difco 055-B5) at 25 μg/ml to induce NF-kB expression. The percent reduction in NF-kB induced expression was recorded as an indicator of NF-kB inhibition. The process has been described in detail by Liberg et al., Br J Cancer 81(6): 981-988, 1999, which is herein incorporated by reference in its entirety.

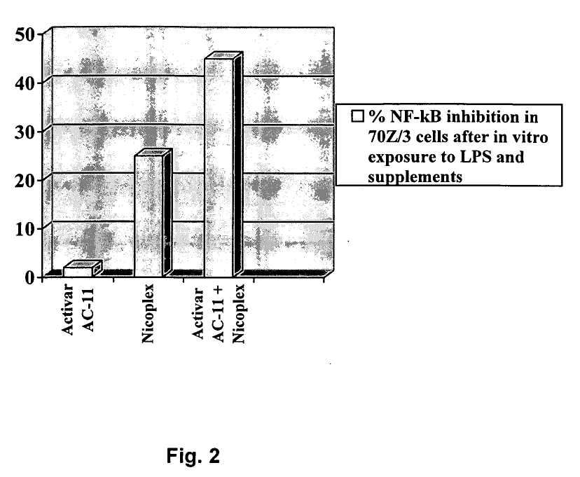

FIG. 2 shows the NF-kB inhibition after lipopoluysaccharride in vitro stimulation of 70Z/3 mouse lymphoma cells. As shown, the supplement containing the combination of 12 μg/ml of Activar AC-11™ and 136 μg/ml of Nicoplex® achieved an unexpected profound enhancement on NF-kB inhibition greater than a mere combination of each individual's effect. This result demonstrated a strong synergetic effect obtained in the composition containing carotenoids, nicotinamide, zinc and the water soluble extract material of the Uncaria species.

### EXAMPLE 3

### In Vitro Analysis of Tumor Cell Growth Inhibition

The commercial products Activar AC-11™, Nicoplex®, and a combination of Activar AC-11™ (566 mg) and Nicoplex® (50 mg) were used in the test.

Human leukemic HL-60 cells were grown on RPMI 1640 medium fortified with 10% fetal calf serum in a 5% carbon dioxide in a 37° C. incubator with 80% humidity. The cells used in all experiments were first cultured for 2 days at an initial density of 2×10⁵ prior to use in the in vitro assays. This resulted in exponential growth stage and cell viability of 95% by trypan blue exclusion. Next, the anti-proliferative capacity of the supplements were determined by colorimetric MTT (3-(4,5-dimethylthiazol-2-yl)-2,5-diphenyltetrazolium bromide) assay as described by Schweitzer et al. Exper Hematol 21: 573-578, 1993, which is herein incorporated by reference in its entirety.

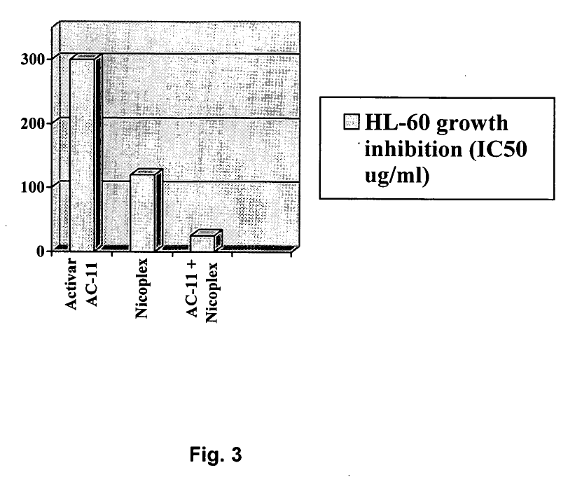

More specifically, 10 μl of serial duplicate dilutions of the supplements were added to 190 μl of cells (0.05×10⁶) in 96-well, flat bottomed plates (Corning, N.Y.) to give the final serial concentrations of 0-500 μg/ml for Nicoplex®, of 0-1000 μg/ml Activar AC-11™ and of 0-60 μg/ml for the Acitvar AC-11™+Nicoplex® combination. Plates were incubated for 72 hours at 37° C., pulsed with 20 μl MTT (5 mg/ml, Sigma), then incubated for an additional 3 hours at 37° C. Reduced MTT was measured spectrophotometrically with an automated plate reader at 540 nm after lysis of cells with 150 μl of DMSO and 25 μl of 0.1 M glycine buffer (pH=10.5). IC50 (inhibitory concentration 50%) values for each supplement were calculated from the serial dilution curve plots and recorded in FIG. 3.

As shown, Nicoplex®, which is the combination of carotenoids, nicotinamide, zinc materials, had a strong effect in inhibition of the HL-60 growth. Furthermore, the combination of Activar AC-11™ and Nicoplex® achieved a profound enhancement in inhibition of the HL-60 growth than Activar AC-11™ or Nicoplex® individually. Similar to the improvement on NF-kB inhibition as illustrated in Example 2, a strong synergetic effect is obtained in inhibition of tumor cell growth using the composition containing carotenoids, nicotinamide, zinc and the water soluble extract material of the Uncaria species.

While the invention has been disclosed in connection with certain preferred embodiments, this should not be taken as a limitation to all of the provided details. Modifications and variations of the described embodiments may be made without departing from the spirit and scope of the invention, and other embodiments should be understood to be encompassed in the present disclosure as would be understood by those of ordinary skill in the art. All references cited hereinbefore are hereby incorporated by references in their entireties.

1. A supplement for administering to a human or other mammals consisting essentially of:
(a) a carotenoid material;
(b) a nicotinamide material;
(c) a zinc source material; and
(d) a water soluble extract material of an Uncaria species.

2. The supplement of claim 1, wherein said nicotinamide material is at least one selected from the group consisting of nicotinamide, niacin, tryptophane, nicotinamide-adenine dinucleotide (NAD), reduced form of NAD (NADH), NAD phosphate (NADP), reduced form of NADP (NADPH) and combination thereof.

3. The supplement of claim 1, wherein said carotenoid material is at least one selected from the group consisting of alpha carotene, beta carotene, gamma carotene, lycopene and combination thereof.

4. The supplement of claim 1, wherein said zinc source material is one or more zinc salts.

5. The supplement of claim 1, wherein said supplement is in a form for oral administration.

6. The supplement of claim 1, wherein said supplement is in a form for parenteral administration.

7. A liquid supplement for administering to a human or other mammals consisting essentially of:
(a) a nicotinamide material;
(b) a zinc source material;
(c) a water soluble extract material of an Uncaria species; and
(d) an aqueous medium.

8. The liquid supplement of claim 7, wherein said nicotinamide material is at least one selected from the group consisting of nicotinamide, niacin, tryptophane, NAD, NADH, NADP, NADPH and combination thereof

9. The liquid supplement of claim 7, wherein said zinc source material is one or more zinc salts.

10. The liquid supplement of claim 7 further consisting of a carotenoid material.

11. The liquid supplement of claim 10, wherein said carotenoid material is at least one selected from the group consisting of alpha carotene, beta carotene, gamma carotene, lycopene and combination thereof.

12. The liquid supplement of claim 7, wherein said liquid supplement has a pH in a range from about 7.5 to about 9.0.

13. The liquid supplement of claim 7, wherein said liquid supplement is in a form of a nutritional drink.

14. A method of treating a mammal comprising administering to said mammal a supplement consisting essentially of a carotenoid material, a nicotinamide material, a zinc source material, and a water soluble extract material of an Uncaria species, said materials being administered to said mammal in daily dosage of amounts effective, in combination, to improve resistance to DNA damage, enhance DNA repair capacity, stimulate immune cell function, and inhibit tumor cell growth.

15. The method of claim 14, wherein said daily dosage comprises from about 50 to 150 mg of carotenoid, from about 50 to about 150 mg of nicotinamide, from about 5 to about 50 mg of a zinc salt, from about 100 to about 1000 mg of the water soluble extract of the Uncaria species.

16. The method of claim 14, wherein said mammal is a human.
